(19) United States
(12) Patent Application Publication
(10) Pub. No.: US 2023/0255705 A1
(43) Pub. Date: Aug. 17, 2023

(54) SYSTEM AND METHOD FOR CALIBRATING A SURGICAL INSTRUMENT

(71) Applicant: Covidien LP, Mansfield, MA (US)

(72) Inventors: Colin H. Murphy, Cambridge, MA (US); Andrew W. Zeccola, Allston, MA (US); Paul M. Loschak, Somerville, MA (US)

(21) Appl. No.: 18/108,777

(22) Filed: Feb. 13, 2023

Related U.S. Application Data
(60) Provisional application No. 63/310,777, filed on Feb. 16, 2022.

(51) Int. Cl. A61B 34/32; A61B 34/20

(52) U.S. Cl. CPC A61B 34/32; A61B 34/20; A61B 2034/2055

(57) ABSTRACT

A surgical robotic system includes a robotic arm having a camera that is configured to output a video stream. The system also includes a surgical instrument coupled to the same or another robotic arm and a controller configured to receive the video stream from the camera and to calibrate the surgical instrument based on the video stream.

### CROSS-REFERENCE TO RELATED APPLICATIONS

This application claims the benefit of and priority to U.S. Provisional Patent Application No. 63/310,777 filed Feb. 16, 2022. The entire disclosure of the foregoing application is incorporated by referenced herein.

### BACKGROUND

Surgical robotic systems may include a surgeon console controlling one or more surgical robotic arms, each having a surgical instrument having an end effector (e.g., forceps or grasping instrument). In operation, the robotic arm is moved to a position over a patient and the surgical instrument is guided into a small incision via a surgical access port or a natural orifice of a patient to position the end effector at a work site within the patient's body.

Due to remote operation of surgical robotic instruments, actuation of the instrument needs to precisely reflect the movement commands input remotely at a surgical console. Thus, prior to using instruments, the robotic systems calibrate the instruments.

### SUMMARY

According to one embodiment of the present disclosure, a surgical robotic system is disclosed. The surgical robotic system includes a robotic arm having a camera that is configured to output a video stream. The system also includes a surgical instrument coupled to the same or another robotic arm and a controller configured to receive the video stream from the camera and to calibrate the surgical instrument based on the video stream.

Implementations of the above embodiment may include one or more of the following features. According to one aspect of the above embodiment, the controller may be further configured to determine whether the surgical instrument is detected by the camera. The controller may be also configured to determine whether the surgical instrument is detected by the camera based on a distance of the surgical instrument from the camera or whether the surgical instrument is in focus. The controller may be also configured to move the surgical instrument and/or the camera until the surgical instrument is detected by the camera. The surgical instrument may include an end effector having at least one degree of freedom. The controller may be further configured to identify a type of the surgical instrument. The controller may be also configured to identify the type of the surgical instrument from the video stream. The controller may be further configured to select a calibration routine based on the type of the surgical instrument. The controller may be also configured to calibrate the surgical instrument by moving the end effector in the at least one degree of freedom to a calibration position based on the calibration routine and receiving the video stream of the end effector being moved to the calibration position. The video stream may include positional information of the end effector. The controller may be also configured to calibrate the surgical instrument by correlating the positional information to the calibration position and calculating a calibration factor based on a difference between the positional information and the calibration position.

According to another embodiment of the present disclosure, a method for calibrating a surgical instrument is disclosed. The method includes transmitting from a camera to a controller a video stream of the surgical instrument and calibrating, at the controller, the surgical instrument based on the video stream.

Implementations of the above embodiment may include one or more of the following features. According to one aspect of the above embodiment, the method may further include determining, at the controller, whether the surgical instrument is detected by the camera based on a distance of the surgical instrument from the camera or whether the surgical instrument is in focus. The method may further include moving at least one of the surgical instrument or the camera until the surgical instrument is detected by the camera. The method may also include identifying, at the controller, a type of the surgical instrument from the video stream and selecting, at the controller, a calibration routine based on the type of the surgical instrument. The method may additionally include moving an end effector of the surgical instrument in at least one degree of freedom to a calibration position and receiving the video stream of the end effector being moved to the calibration position, the video stream includes positional information of the end effector. The method may further include correlating the positional information to the calibration position and calculating a calibration factor based on a difference between the positional information and the calibration position.

According to a further embodiment of the present disclosure, a surgical robotic system is disclosed. The surgical robotic system includes a having a camera configured to output a video stream. The system also includes a surgical instrument with an end effector, and a controller configured to receive the video stream from the camera and to calibrate the end effector based on the video stream.

Implementations of the above embodiment may include one or more of the following features. According to one aspect of the above embodiment, the controller may be further configured to determine whether the surgical instrument is detected by the camera based on a distance of the surgical instrument from the camera or whether the surgical instrument is in focus; and move the surgical instrument and/or the camera until the surgical instrument is detected by the camera. The controller may be further configured to identify a type of the surgical instrument from the video stream. The controller may be also configured to select a calibration routine based on the type of the surgical instrument. The controller may be also configured to calibrate the surgical instrument by moving the end effector in the at least one degree of freedom to a calibration position based on the calibration routine and receiving the video stream of the end effector being moved to the calibration position, the video stream may include positional information of the end effector. The controller may be also configured to calibrate the surgical instrument by correlating the positional information to the calibration position and calculating a calibration factor based on a difference between the positional information and the calibration position.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a perspective view, with parts separated, of an instrument drive unit and a surgical instrument according to an embodiment of the present disclosure;

FIG. 6 is a top, perspective view of an end effector, according to an embodiment of the present disclosure, for use in the surgical robotic system of FIG. 1;

FIG. 7 is a schematic view of a calibration system including an endoscopic camera and a surgical instrument according to an embodiment of the present disclosure; and

FIG. 8 is a flow chart of a method for calibrating the surgical instrument using the endoscopic camera of FIG. 7 according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “proximal” refers to the portion of the surgical robotic system and/or the surgical instrument coupled thereto that is closer to a base of a robot, while the term “distal” refers to the portion that is farther from the base of the robot.

As will be described in detail below, the present disclosure is directed to a surgical robotic system, which includes a surgeon console, a control tower, and one or more mobile carts having a surgical robotic arm coupled to a setup arm. The surgeon console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

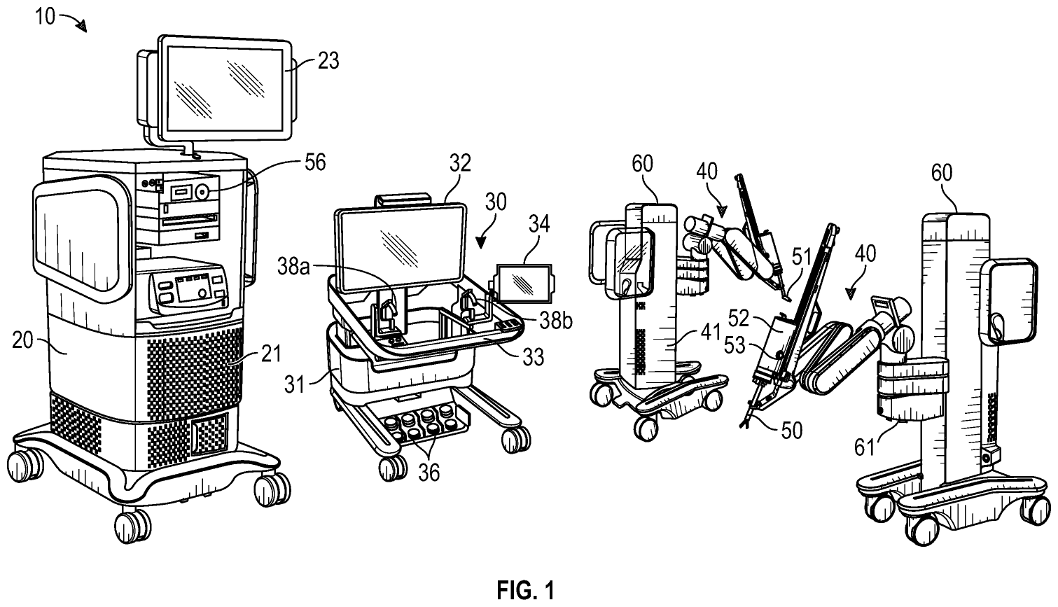

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 removably coupled thereto. The robotic arms 40 is also coupled to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image and output the processed video stream.

The surgeon console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

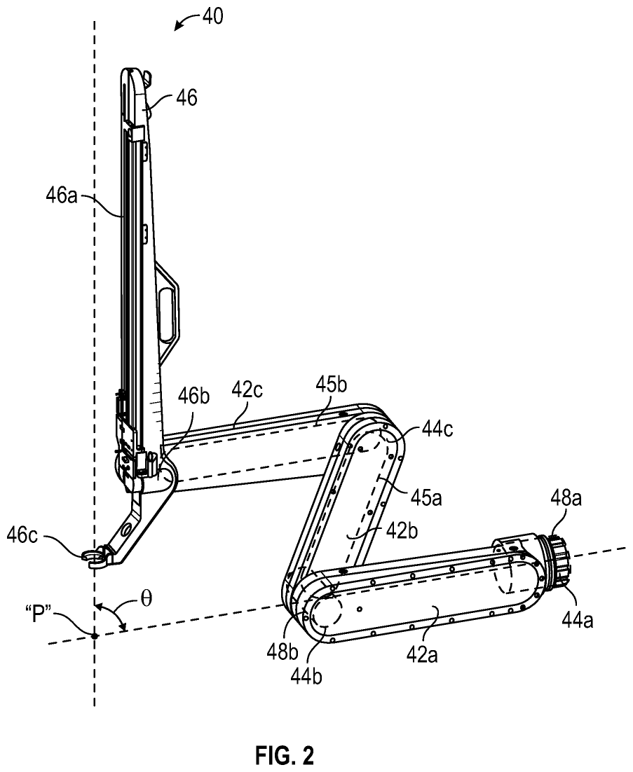

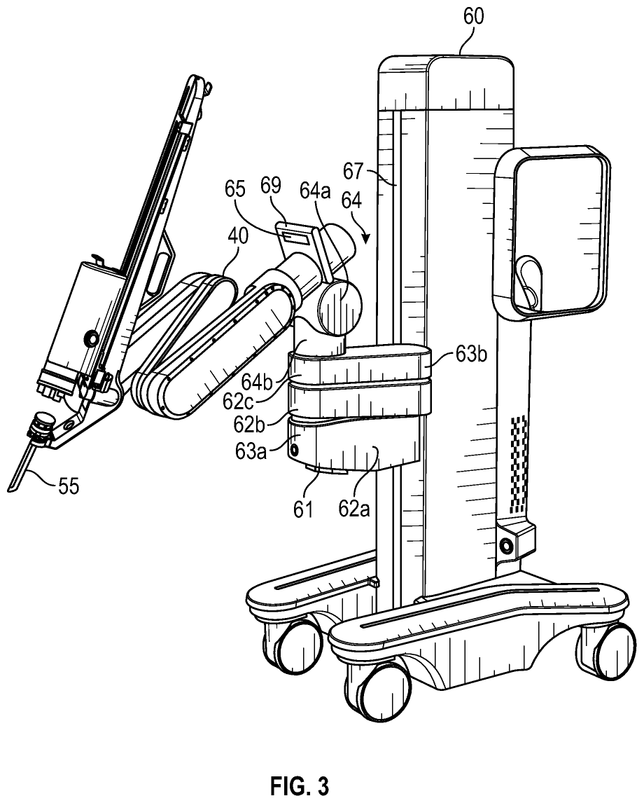

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

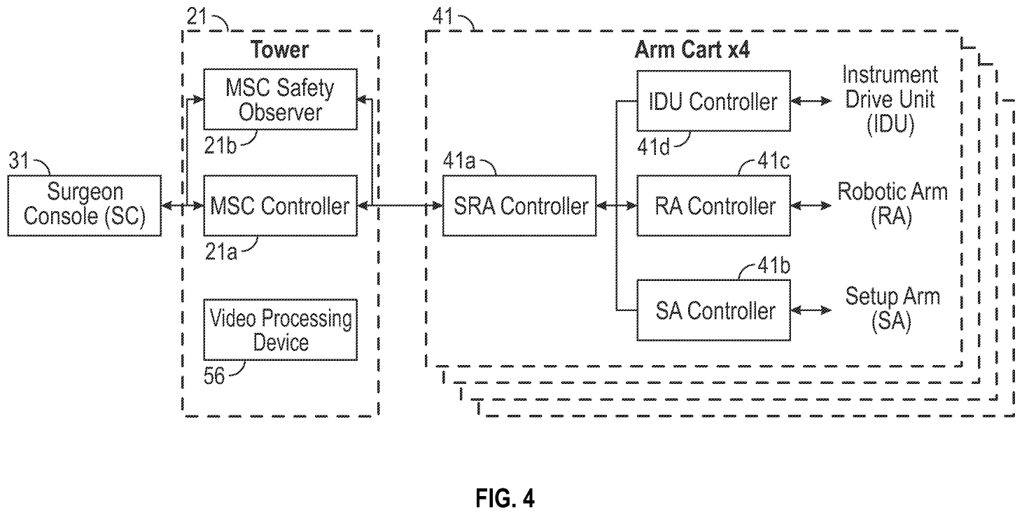

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

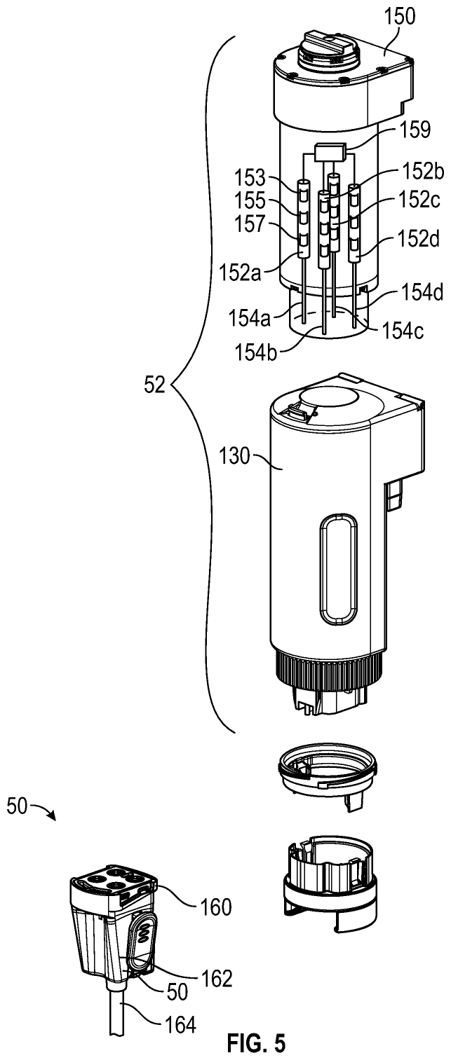

With reference to FIG. 5, the IDU 52 is shown in more detail and is configured to transfer power and actuation forces from its motors 152a, 152b, 152c, 152d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

The IDU 52 includes a motor pack 150 and a sterile barrier housing 130. Motor pack 150 includes motors 152a, 152b, 152c, 152d for controlling various operations of the instrument 50. The instrument 50 is removably couplable to IDU 52. As the motors 152a, 152b, 152c, 152d of the motor pack 150 are actuated, rotation of the drive transfer shafts 154a, 154b, 154c, 154d of the motors 152a, 152b, 152c, 152d, respectively, is transferred to the drive assemblies of the instrument 50.

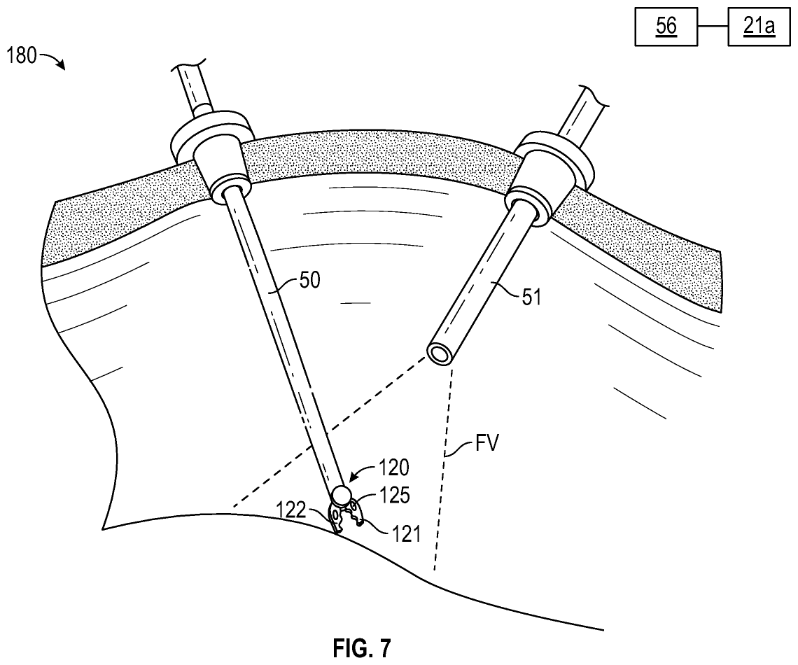

The instrument 50 is configured to transfer rotational forces/movement supplied by the IDU 52 (e.g., via the motors 152a, 152b, 152c, 152d of the motor pack 150) into longitudinal movement or translation of the cables or drive shafts to effect various functions of an end effector 120 (FIG. 7).

Each of the motors 152a, 152b, 152c, 152d includes a current sensor 153, a torque sensor 155, and an encoder sensor 157. For conciseness only operation of the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by the motor 152a. The encoder sensor 157 may be any device that provides a sensor signal indicative of the number of rotations of the motor 152a, such as a mechanical encoder or an optical encoder. Parameters which are measured and/or determined by the encoder sensor 157 may include speed, distance, revolutions per minute, position, and the like. The sensor signals from sensors 153, 155, 157 are transmitted to the IDU controller 41d, which then controls the motors 152a, 152b, 152c, 152d based on the sensor signals. In particular, the motors 152a, 152b, 152c, 152d are controlled by an actuator controller 159, which controls torque outputted and angular velocity of the motors 152a, 152b, 152c, 152d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

With reference to FIG. 5, instrument 50 includes an adapter 160 having a housing 162 at a proximal end portion thereof and an elongated shaft 164 that extends distally from housing 162. Housing 162 of instrument 50 is configured to selectively couple to IDU 52 of robotic, to enable motors 152a, 152b, 152c, 152d of IDU 52 to operate the end effector 120 of the instrument 50. Housing 162 of instrument 50 supports a drive assembly that mechanically and/or electrically cooperates with motors 152a, 152b, 152c, 152d of IDU 52. Drive assembly of instrument 50 may include any suitable electrical and/or mechanical component to effectuate driving force/movement.

The surgical instrument also includes an end effector 120 coupled to the elongated shaft 164. The end effector 120 may include any number of degrees of freedom allowing the end effector 120 to articulate, pivot, etc., relative to the elongated shaft 164. The end effector 120 may be any suitable surgical end effector configured to treat tissue, such as a dissector, grasper, sealer, stapler, etc.

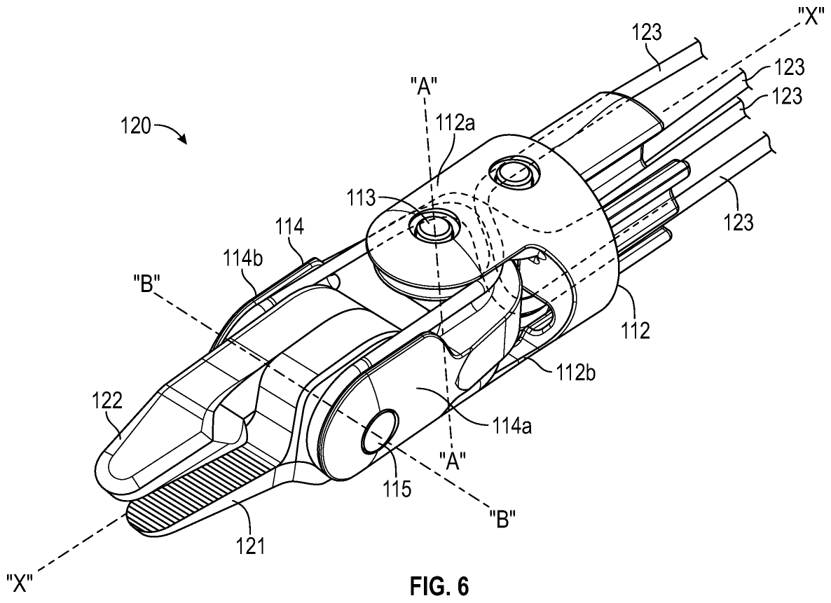

As shown in FIG. 6, the end effector 120 may include a pair of opposing jaws 121 and 122 that are movable relative to each other. In embodiments, the end effector 120 may include a proximal portion 112 having a first pin 113 and a distal portion 114. The end effector 120 may be actuated using a plurality of cables 123 routed through proximal and distal portions 112 and 114 around their respective pulleys 112a, 112b, 114a, 114b, which are integrally formed as arms of the proximal and distal portions 112 and 114. In embodiments, the end effector 120, namely, the distal portion 114 and the jaws 121 and 122, may be articulated about the axis “A-A” to control a yaw angle of the end effector with respect to a longitudinal axis “X-X”. The distal portion 114 includes a second pin 115 with a pair of jaws 121 and 122 pivotably coupled to the second pin 115. The jaws 121 and 122 configured to pivot about an axis “B-B” defined by the second pin 115 allowing for controlling a pitch angle of the jaws 121 and 122 as well as opening and closing the jaws 121 and 122. The yaw, pitch, and jaw angles are controlled by adjusting the tension and/or length and direction (e.g., proximal or distal) of the cables 123. Thus, the end effector 120 may have three degrees of freedom, yaw, pitch, and jaw angle between jaws 121 and 122.

With reference to FIG. 7, a calibration subsystem 180 includes the camera 51 and the video processing device 56 as well as the controller 21a. Calibration may be performed outside and/or inside the patient while the end effector 120 is within a field of view “FV” of the camera 51. The video processing device 56 is configured to receive video stream from the camera 51 and may transmit the video stream to another computing device, e.g., controller 21a, for further processing. The controller 21a may utilize machine learning or other image processing techniques to identify, track, and/or calibrate the surgical instrument 50, the end effector 120, and/or the jaws 121 and 122.

Portions of the method of the present disclosure are described as being performed by one or more individual controllers and/or computing devices. Such description is merely exemplary, and functionality, i.e., software instructions, may be performed or executed by any number or combination of controllers in any suitable manner.

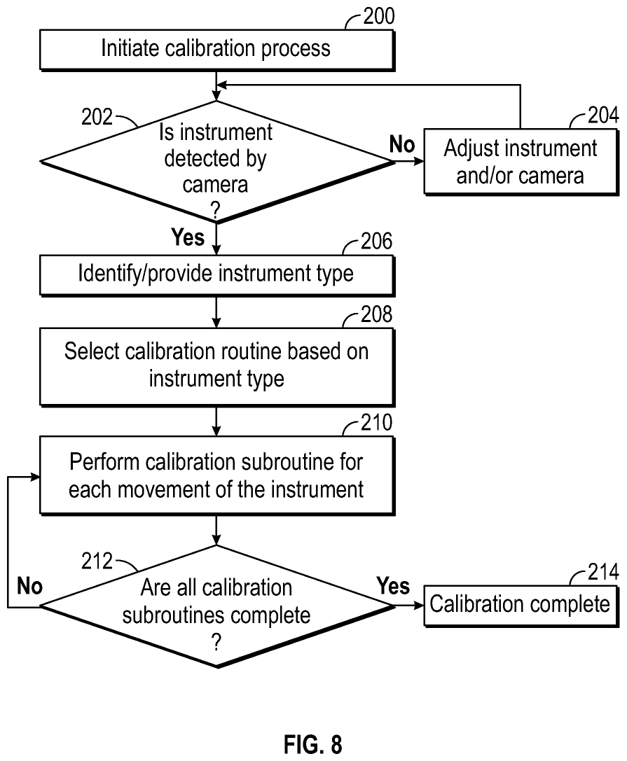

With reference to FIG. 8, a flow chart of a calibration method includes initiating the calibration process at step 200. Calibration may be initiated manually by the user, e.g., toggling GUI of the display 23 or of a display of the robotic arm 40. In embodiments, calibration may be done automatically after the instrument 50 is coupled to the IDU 52, which establishes mechanical and/or electrical connections therebetween enabling actuation of the end effector 120 and the instrument 50 by the IDU 52 as well as movement along the sliding mechanism 46a.

At step 202, the controller 21a is configured to determine whether the end effector 120 is properly viewable by the camera 51. This may include determining whether the end effector 120 is within the field of view “FV” of the camera 51 e.g., disposed within central area of the objective. Determination of the end effector 120 being properly viewable may also include determining whether the end effector 120 is located at a desired distance from the camera 51 and/or whether the end effector 120 is in focus. Distance may be determined using depth mapping or any other suitable image processing technique. The end effector 120 may be identified and tracked using a computer vision algorithm derived from machine learning techniques, such as a deep neural network trained to recognize and identify type, position, orientation, operational state of the surgical instrument 50, the end effector 120, and/or the jaws 121 and 122 in the field of view “FV” of the camera 51. In embodiments, the surgical instruments 50 may include fiducial markers 125 (FIG. 7), such as dots, patterns, etc. that are used to identify and/or track end effector 120 of the surgical instruments 50. In other aspects, the user or clinician is prompted by the controller 21a to move the camera 51 and/or instrument 50 until the end effector 120 of the instrument is in the field of view.

If the end effector 120 is not properly viewable by the camera 51, then the controller 21a adjusts the position of the instrument 50 and/or the camera 51 at step 204. In other embodiments, if end effector 120 is not in the field of view of the camera 51, the controller 21a provides a prompt to the user to move the camera 51 and/or instrument 50 until the end effector 120 of the instrument is in the field of view. The controller 21a may command the robotic arm 40 holding the instrument 50 and/or the camera 51 to move one or both of the instrument 50 and/or the camera 51 until the instrument 50 is properly viewable by the camera 51. In embodiments, imaging or operational parameters of the camera 51 may also be adjusted by the controller 21a to enhance the video stream, such as adjusting exposure, zoom, contrast, etc. The step 204 may be repeated until the controller 21a confirms that the end effector 120 is viewable by the camera 51.

At step 206, the controller 21a identifies the type of the instrument 50. This may be done by communicating with the instrument 50, e.g., interrogating a storage device associated with the instrument 50 storing identifying information, model, serial number etc. In embodiments, the controller 21a may identify the type of the instrument 50 from the video stream using unique features of the instrument 50, e.g., the fiducial marker 125 or an indicator, shape of the jaws 121 and 122, etc. In other aspects, the controller 21a prompts the user or clinician to input the type of the tool or end effector 120. The controller 21a at step 208, in response to a user or clinician input selects a calibration routine.

At step 208, the controller 21a selects a calibration routine based on the type of the instrument 50. Different types of instruments have different types of end effectors 120, e.g., different number of degrees of freedom, length of jaw members, etc., which utilize different movements for calibration, e.g., to determine movement limits, center position, etc. In embodiments, the end effector 120 may be calibrated in its yaw, pitch, and jaw angle and the instrument 50 may also be rotated about its longitudinal axis “X-X”. In other aspects, the controller 21a prompts the user or clinician to input the tool and/or calibration routine needed for calibration. The controller 21a at step 208, in response to a user or clinician input selects a calibration routine.

Each of the calibration routines may include a plurality of subroutines, each of which is used to calibrate each of the movements of the end effector 120. Calibration is performed for each of the degrees of freedom at step 210, e.g., yaw, pitch, and jaw angle between jaws 121 and 122. This may be accomplished by moving the end effector 120 about each of the axes “A-A” and “B-B” as well as opening the jaws 121 and 122 between two limits of corresponding motion range. During yaw calibration, the end effector 120 is pivoted about the “A-A” axis in either direction until the end effector 120 reaches mechanical end limit. Pitch calibration is performed similarly to yaw calibration, except that the end effector 120 is pivoted about the “B-B” axis. Jaw angle calibration is performed by moving, i.e., opening, both jaws 121 and 122 until each of the jaws 121 and 122 reach a mechanical end limit.

During calibration, while the end effector 120 or one of its components, e.g., the jaws 121 and 122, are moved, the camera 51 provides the video feed to the controller 21a, which then determines the distance traveled during movement of the end effector 120 and/or the jaws 121 and 122.

Each calibration subroutine includes the controller 21a commanding the jaw members 121 and 122, i.e., through the IDU controller 41d, to be moved (e.g., pivoted, articulated, opened, etc.) from one extreme or limit, i.e., calibration position, to another. During this movement, the camera 51 continuously provides a video stream of the end effector 120 and in particular the position of the end effector 120 at each of the limits. The controller 21a receives the video stream and correlates the video stream, including video positional information (i.e., traveled distance) with kinematics positional information (i.e., commanded movement). The difference between the positional information of each of the calibration subroutine to calculate a calibration factor, which is then applied to inverse kinematics moving the end effector 120.

At step 212, the controller 21a determines whether each of the calibration subroutines have been completed. If there are remaining subroutines, the controller 21a proceeds through each of them. If all of the subroutines are complete, then at step 214 the controller 21a indicates that calibration is complete, and the instrument 50 is ready for teleoperation through the surgical console 30.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a camera that is configured to output a video stream;
a surgical instrument; and
a controller configured to receive the video stream from the camera and to calibrate the surgical instrument based on the video stream.

2. The surgical robotic system according to claim 1, wherein the camera is coupled to a robotic arm and the surgical instrument is coupled to the same or a different robotic arm as the camera.

3. The surgical robotic system according to claim 1, wherein the controller is further configured to determine whether the surgical instrument is detected by the camera.

4. The surgical robotic system according to claim 3, wherein the controller is further configured to determine whether the surgical instrument is detected by the camera based on a distance of the surgical instrument from the camera or whether the surgical instrument is in focus.

5. The surgical robotic system according to claim 3, wherein the controller is further configured to move at least one of the surgical instrument or the camera until the surgical instrument is detected by the camera.

6. The surgical robotic system according to claim 1, wherein the surgical instrument includes an end effector including at least one degree of freedom and the controller is further configured to identify a type of the surgical instrument.

7. The surgical robotic system according to claim 6, wherein the controller is further configured to identify the type of the surgical instrument from the video stream.

8. The surgical robotic system according to claim 6, wherein the controller is further configured to select a calibration routine based on the type of the surgical instrument.

9. The surgical robotic system according to claim 8, wherein the controller is further configured to calibrate the surgical instrument by:
moving the end effector in the at least one degree of freedom to a calibration position based on the calibration routine;
receiving the video stream of the end effector being moved to the calibration position, the video stream including positional information of the end effector;
correlating the positional information to the calibration position; and
calculating a calibration factor based on a difference between the positional information and the calibration position.

10. A method for calibrating a surgical instrument, the method comprising:
transmitting from a camera to a controller a video stream of the surgical instrument; and
calibrating, at the controller, the surgical instrument based on the video stream.

11. The method according to claim 10, further comprising:
determining, at the controller, whether the surgical instrument is detected by the camera based on a distance of the surgical instrument from the camera or whether the surgical instrument is in focus.

12. The method according to claim 11, further comprising:
moving at least one of the surgical instrument or the camera until the surgical instrument is detected by the camera.

13. The method according to claim 11, further comprising:
identifying, at the controller, a type of the surgical instrument from the video stream; and
selecting, at the controller, a calibration routine based on the type of the surgical instrument.

14. The method according to claim 13, further comprising:
moving an end effector of the surgical instrument in at least one degree of freedom to a calibration position; and
receiving the video stream of the end effector being moved to the calibration position, the video stream including positional information of the end effector.

15. The method according to claim 14, further comprising:
correlating the positional information to the calibration position; and
calculating a calibration factor based on a difference between the positional information and the calibration position.

16. A surgical robotic system comprising:
a first robotic arm including a camera configured to output a video stream;
a second robotic arm including a surgical instrument having an end effector; and
a controller configured to receive the video stream from the camera and to calibrate the end effector based on the video stream.

17. The surgical robotic system according to claim 16, wherein the controller is further configured to:
determine whether the surgical instrument is detected by the camera based on a distance of the surgical instrument from the camera or whether the surgical instrument is in focus; and
move at least one of the surgical instrument or the camera until the surgical instrument is detected by the camera.

18. The surgical robotic system according to claim 16, wherein the controller is further configured to identify a type of the surgical instrument from the video stream.

19. The surgical robotic system according to claim 18, wherein the controller is further configured to select a calibration routine based on the type of the surgical instrument.

20. The surgical robotic system according to claim 19, wherein the controller is further configured to calibrate the surgical instrument by:
moving the end effector to a calibration position based on the calibration routine;
receiving the video stream of the end effector being moved to the calibration position, the video stream including positional information of the end effector;
correlating the positional information to the calibration position; and
calculating a calibration factor based on a difference between the positional information and the calibration position.
